(19) United States
(12) Patent Application Publication
(10) Pub. No.: US 2005/0020909 A1
(43) Pub. Date: Jan. 27, 2005

(54) Display device for surgery and method for using the same

(76) Inventors: Jose Luis Moctezuma de la Barrera, Freiburg (DE); Donald W. Malackowski, Schoolcraft, MI (US)

Correspondence Address: MCCRACKEN & FRANK LLP, 200 W. ADAMS STREET, SUITE 2150, CHICAGO, IL, 60606, US

(21) Appl. No.: 10/617,077

(22) Filed: Jul. 10, 2003

(51) Int. Cl. A61B 5/05; A61B 19/00

(52) U.S. Cl. 600/424; 606/130

(57) ABSTRACT

A display unit for use with a surgical navigation system that includes a body; a screen capable of displaying data; a communications link capable of sending and receiving data to and from the surgical navigation system; and an attachment device to attach the display unit to a body of a surgical instrument to be manipulated relative to a patient's anatomy, a tracking unit associated with the surgical instrument that can be tracked by the surgical navigation system; where the screen can display information received from the surgical navigation system. A method for using the display unit attached to a surgical instrument is also disclosed.

### FIELD OF THE INVENTION

This invention relates to display devices for use with a surgical navigation system. More particularly this invention relates to display devices that can directly provide the surgeon with information without the need to look away from the point of surgery.

### BACKGROUND ART

In recent years, there has been increased use by surgeons of computer assisted surgical navigation devices to assist them in guiding a surgical instrument relative to a patient's anatomy. Typical optical surgical navigation systems and devices use small light emitting diodes (LED's) and sensors for these LED's to determine the position of a surgical tool relative to a patient's anatomy. A typical surgical tool that can be tracked by the surgical navigation system includes multiple LED's. In addition to optical based systems, there are magnetic based systems that use magnets and magnetic sensors, inertial based systems that use the earth's gravitational pull, robotic systems, and combination systems some or all of these system elements or other tracking systems.

Typical optical surgical navigation systems utilize three separately spaced apart detectors and associated computer software to determine the position of each of the LED's on a surgical device or instrument. In these systems, the LED light sources are small relative to the distance and volume of the space and therefore are considered as point sources of light. Typical LED's emit light within the infrared spectrum, however, light of other wavelengths can be used, including light within the visible spectrum. When the LED point sources are detected by multiple light detection devices, the exact x, y, z location of each LED on the surgical instrument or tool can be determined. The determination of the location of these LED's for a properly calibrated tool can determine both the position of that tool and the orientation of that tool within a three-dimensional surgical space or volume.

The typical surgical navigation system displays the location of the tracked tool or instrument on a display monitor within the surgical arena associated with the surgical navigation system. The typical display on these monitors shows a portion of the patient's anatomy based a pre-surgical scan of that anatomy using well known devices such as such as CT or MRI scanning systems. The display also shows the location and orientation of the surgical tool overlaid on the patient's anatomy. The display is updated as the surgeon moves the tool. While these displays assist the surgeon in placing the tool in the appropriate location and orientation, the location of these displays require the surgeon to look away from the point of surgery to the monitor or display device during the procedure. Therefore, it is desirable to have a display unit that is associated with a surgical instrument or tool such that the surgeon can easily see and utilize the information displayed on the display unit without the need to look away from the point of surgery.

### SUMMARY OF THE INVENTION

The present invention comprises a display unit for use with a surgical instrument to be manipulated relative to a patient's anatomy having a surgical instrument body and a tracking unit associated with the surgical instrument capable of communicating with a surgical navigation system. The display unit includes a communication link to communicate with the surgical navigation system, a screen to display information received from the surgical navigation system, and an attachment device to attach the display unit to be attached to the surgical instrument body.

An additional embodiment of the present invention comprises a display unit for use with a surgical navigation system that includes a body, a screen capable of displaying data, a tracking unit associated with the body that can be tracked by the surgical navigation system, a communication link capable of sending and receiving data to and from the surgical navigation system and an attachment device to attach the display unit to a body of a surgical instrument to be manipulated relative to a patient's anatomy. The screen of this display unit can display information relating to the position of the instrument relative to the patient's anatomy.

A further embodiment of the present invention comprises a surgical tool having a surgical tool body, a tool tip, a transceiver capable of sending and receiving data to and from the surgical navigation system associated with the tool body, a display unit associated with the tool body and a tracking unit associated with the tool body such that the surgical tool can be tracked by a surgical navigation system. The display unit has a screen capable of displaying the position of the tool tip relative to a predetermined position.

A still further embodiment of the present invention comprises a method for positioning a tool tip of a surgical tool relative to a predetermined position in a surgical site, which comprises the steps of first viewing the position of the tool tip relative to the predetermined position on a display unit attached to the surgical tool. The display unit receives data about the location of the surgical tool tip from a surgical navigation system and guiding the tool tip to the predetermined position while simultaneously viewing both the predetermined position and the display unit.

Another embodiment of the present invention comprises a method for positioning a surgical device relative to a patient's anatomy, which comprises the steps of viewing information representing the position of the surgical device relative to the patient's anatomy on a display unit attached to the surgical device, where the display unit receives data about the location of the surgical device and data about the location of the patient's anatomy from a surgical navigation system. The method also includes the step of manipulating the surgical device relative to the patient's anatomy using data displayed on the display unit while viewing the both the patient's anatomy and the display unit.

Other aspects and advantages of the present invention will become apparent upon consideration of the following detailed description.

### BRIEF DESCRIPTION OF THE FIGURES

FIG. 1 is a perspective view of a surgical drill that includes an attached display unit that includes tracking elements;

FIG. 2 is an exploded perspective view showing the surgical drill separated from the display unit;

FIG. 3 shows a perspective view of the display unit shown in FIG. 2;

FIG. 4 shows a perspective view of the display unit of FIG. 2 taken from the side opposite FIG. 3;

FIG. 5 is a plan view of an alternative embodiment of the display unit of FIG. 2;

FIG. 6 is a schematic view of the graphical display of the display unit of FIG. 2 showing the tool tip position on the display device when the tool is not in the predetermined position;

FIG. 7 is a view similar to that of FIG. 6 showing the display where the tool tip is in the predetermined position and also showing the display where the orientation of the tool is not in the correct orientation;

FIG. 8 is a view similar to that of FIG. 6 showing the display where the tool tip is in the predetermined position and also showing the display where the orientation of the tool is in the correct orientation;

FIG. 9 is a view similar to that of FIG. 6 showing the display where the tool tip is in the predetermined position and where the orientation of the tool is in the correct orientation, and showing an additional display to indicate the depth;

FIG. 10 is a perspective view of a surgical drill having a display unit attached to the drill by an alternative attachment means;

FIG. 11 is a flow diagram showing the method of the present invention;

FIG. 12 is a perspective view of a surgical saw having a display unit attached to the saw;

FIG. 13 a schematic view of the graphical display of the display unit of FIG. 12 showing the display when the saw blade is not in the proper position;

FIG. 14 a schematic view similar to that of FIG. 13 showing the display when the saw blade is in the proper position, but not in the proper orientation;

FIG. 15 a schematic view similar to that of FIG. 13 showing the display when the saw blade is in both the proper position and the proper orientation;

FIG. 16 is a schematic representation of a surgical navigation system;

FIG. 17 is perspective view of a further embodiment of the display unit of the present invention;

FIG. 18 is a perspective view of the display unit of FIG. 17 showing the back side;

FIG. 19 is a front view of the display unit of FIG. 17 showing the detail of the display LED array;

FIG. 20 is a perspective view of a still further embodiment of the display unit of the present invention shown mounted to a surgical drill that includes an integral tracking device;

FIG. 21 is a perspective view of a biopsy needle device having a further embodiment of the display unit of the present invention attached;

FIG. 22 is a further embodiment of the display unit of the present invention shown mounted to a surgical drill;

FIG. 23 is a further embodiment of the display unit of the present invention shown mounted to an external fixator device; and

FIG. 24 is view of the external fixator device of FIG. 23 showing the manipulation of the external fixator device to properly align a bone.

### DETAILED DESCRIPTION OF THE PREFERRED EMBODIMENTS

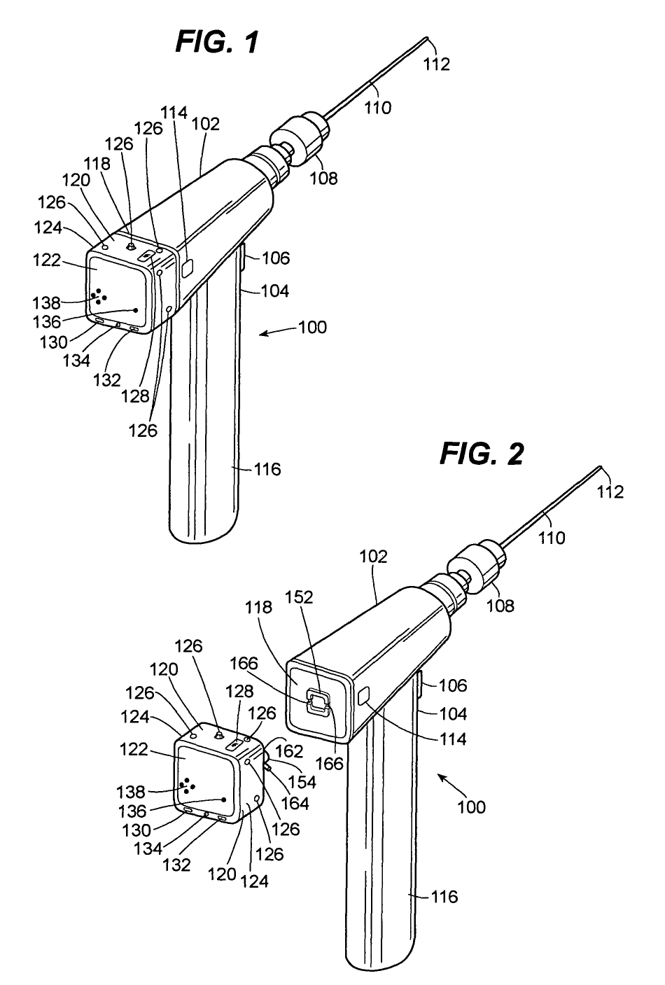

FIGS. 1 and 2 show a surgical power hand tool such as a surgical drill 100 having a tool body 102, a tool handle 104, a control switch 106, a chuck assembly 108, and a drill bit 110 having a tool tip 112. Associated with the tool body 102 is a release button 114 and associated with the tool handle 104 is a battery pack 116. The tool body 102 has a distal end 118.

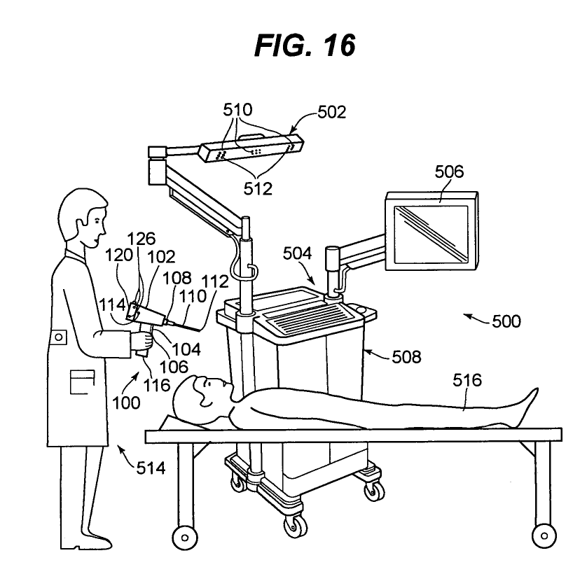

Also associated with the distal end 118 of the tool body 102 is a tracking and display unit 120. FIG. 1 shows the tracking and display unit 120 as attached to the surgical drill 100 while FIG. 2 shows an exploded view with the tracking and display unit 120 removed from the distal end 118 of the tool body 102. The tracking and display unit 120 has a display screen 122, a unit body 124, and a series of LED's 126 spaced on the surface of the unit body 124. The unit body 124 also has a transceiver 128 that can transmit and receive data to and from a surgical navigation system 500 as shown in FIG. 16 using radio or infrared signals. Also located on the unit body 124 below the display screen 122 are a first function button 130 and a second function button 132, the operation of which will be discussed more fully hereinafter. Placed between the first and second function buttons 130 and 132 is a status light 134. Typically the status light 134 will come on for a short period of time after pressing and holding one of the first or second function buttons 130 or 132 to switch on the tracking and display unit 120. Also the status light can be programmed to indicate the other changes in status such as by flashing a pre-designated number of times when a certain function is performed or requested.

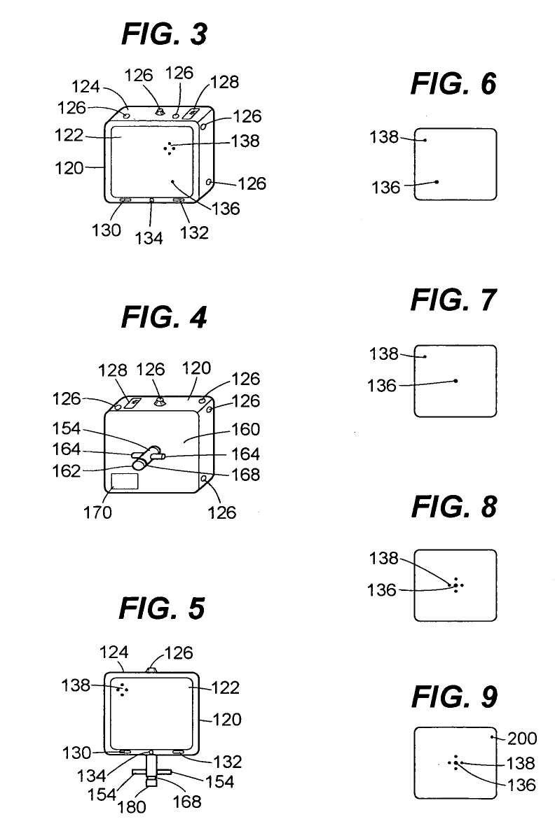

As shown diagrammatically in FIGS. 1 to 3, the display screen 122 can simultaneously display multiple display elements. A tool tip position display 136 can be a single lit pixel or LED that indicates the position of the tool tip 112 relative to a predetermined location. An orientation display 138 is also shown on the display screen 122 as a series of four spaced LED's or pixels in the shape of a cross. These four spaced LED's surround a center unlit LED such that the orientation display 138 can surround the tool tip position display 136. Other representations of the tool tip display 136 and the orientation display 138 are possible so long as the representation effectively communicates the desired information to the user.

Depending on the type and resolution of display screen 122, it is possible that the display unit 120 can display the position and orientation information in other formats. The display screen 122 may also show all or a subset of the information displayed on a monitor 506 associated with the surgical navigation system 500 as shown in FIG. 16. In addition, it is possible that the display screen 122 can show information that is derived from the surgical instrument or tool itself, such as the nature or identity of the tissue, bone, the speed or torque of the tool, and the like. This information may include some of all of the following information: position information; orientation information; current position of a tool or instrument relative to the relevant anatomy of the patient; biomechanical information relative to the anatomy, such as the mechanical axis of a limb or joint; diagnostic images, such as pre-operative scan information, intra-operative scan images, and the like; virtual representations of implants, plates, nails and the like; tool parameters, such as the temperature at the tool tip, torque, rotational speed, and other similar information; and user instructions, warnings, and the like. When the data displayed on the display screen 122 is a subset of the information and images displayed on the monitor 506, the surgeon has the ability to switch view on the display screen 122 to display additional information using one of the function buttons 120 or 132 or touching a designated portion of the display screen 122 in a touch screen mode.

The display screen 122 can be formed from any suitable display device capable of displaying a series of discreet pixels. The screen could be a liquid crystal display device (LCD) screen or it could be an array of LED's such as 12×12 array of LED's. The display screen 122 can be capable of displaying a single color or it can display multiple colors such that the different colors can indicate additional information to the surgeon. For certain procedures the display unit 120 may also have a hole or aperture through the display unit 120 to allow a guide wire or similar device to pass through the display unit 120. As shown in FIGS. 2 and 4, the tracking and display unit 120 is attached to the distal end 118 of the tool body 102 using a docking structure 152. The docking structure 152 is designed to accommodate and lock into place a docking pin 154, which is centered on the back 160 of the unit body 124. The docking pin 154 and the docking structure 152 are formed such that when the tracking and display unit 120 placed against the distal end 118 of the tool body 102 and urged forward, a center post 162 of the docking pin 154 will enter the center of the docking structure 152 and arms 164 attached to the center post 162 will fit within detents 166 so as to firmly orient and fix the tracking and display unit 120 in place on the distal end 118 of the surgical drill 100. Also, the docking pin 154 has an annular groove 168, which cooperates with a locking mechanism (not shown) within the docking structure 152. The release button 114 deactivates this locking mechanism and allows the tracking and display unit 120 to be removed from the surgical drill 100. Although not shown, it is possible to include on the back 160 and on the distal end 118 an electrical interconnect device such that the tracking and display unit 120 can be powered from the battery pack 116. However, it is preferable that tracking and display unit 120 have its own self-contained power source and, accordingly, a battery (not shown) can be inserted into the unit body 124 though a battery door 170. In addition to the docking pin 154 and the docking structure 152, any suitable method of attaching the tracking and display unit 120 to the tool body 102 can be used such as a bayonet coupling or other similar quick release positive locking coupling structure. In addition, the tracking and display unit 120 can be integral with the tool body 102.

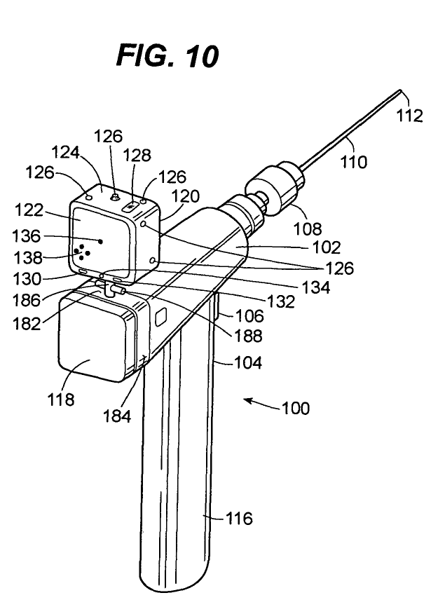

FIGS. 5 and 10 show an alternative way to mount the tracking and display unit 120 onto the surgical drill 100. In the embodiment shown in FIG. 5, a docking pin 180 having a similar structure to the docking pin 154, as discussed with regard to FIGS. 2 and 4, is attached to the bottom of the unit body 124. As shown in FIG. 8, an attachment device 182 is attached to the drill body 102 near the distal end 118. The attachment device 182 includes a band 184 which can be tightened by suitable tightening devices, a docking socket 186, and a release button 188. A docking socket 186 cooperates with the docking pin 180 in the same manner as the docking socket 152 and the docking pin 154. The release button 188 operates in a similar manner to the release button 114.

FIG. 16 is a schematic view of the surgical navigation system 500. As disclosed in published application 2001/0034530, the disclosure of which is incorporated by reference, the surgical navigation system 500 includes a sensor system 502 and a computer system 504. The computer system 504 includes the monitor 506 and a computer (not shown) housed within a computer cart 508. The sensor system includes three CCD cameras 510 to sense the location of the LED's 126. The sensor system 502 also includes a transceiver 512 to communicate with the transceiver 128. The surgeon 514 will view the display screen 122 on the display unit 120 attached to the surgical drill 100 to guide the tool tip 112 to the pre-surgical planned predetermined position on the patient 516. This enables the surgeon 514 to maintain eye contact with the surgical site while at the same time having access to the same information that would be available on the monitor 506 providing better hand eye coordination for the surgeon 514 combined with the benefits of the information available from the surgical navigation system 500.

FIGS. 6 to 9 diagrammatically show the operation of the display screen 122 in cooperation with the surgical navigation system 400 to guide the tool tip 112 into the proper predetermined position and orientation. The surgical navigation system 400 will track the tracking and display unit 120 in a manner similar other tools and devices containing LED's that are visible to the surgical navigation system 400. FIG. 6 shows the tip position display 136 is in the upper left corner of the display 122. This display position of the tool tip position display 136 shows that the tool tip 112 of the surgical drill 100 is not in the correct position that has been determined by preoperative planning. Based on the location of the tip position display 136 as shown in FIG. 6 the tool tip 112 is located to the left and above the predetermined position. The surgeon will move the surgical drill and the tool tip 112 to the correct position. As the surgeon moves the tool tip 112 toward the predetermined position, the tool tip position display 136 will shift toward the center of the display screen 122 to follow the surgeon's movement to the location as shown in FIG. 7. This indicates to the surgeon that the tool tip 112 is in the proper x, y, z position as determined by preoperative planning. Since the surgeon can see the actual location of the predetermined position, in some instances it may only be necessary to provide a two-dimensional position indicator as the surgeon can position the tool tip 112 at the proper depth visually. The tool tip position display can be configured to provide a three-dimensional position indication as well. This can be done using two LED's that will converge as the tool tip 112 reaches the proper depth and x, y position.

As can be easily appreciated, the fact that the tool tip 112 is in the correct x, y, z position does not mean that the drill bit 110 is properly aligned and oriented for the procedure to begin. As shown in FIG. 6, the orientation display 138 shows that the alignment of the drill bit 110 is up and to the left. As the surgeon moves the drill 100 orientation down and to the right to the position diagrammatically shown in FIG. 7, the unaligned tool orientation display 138 will follow the movement down and to the right until such time as tool orientation display 138 surrounds the properly aligned tool tip position 136 to form a cross as shown as shown in FIG. 8. If the display screen 122 is monochromatic, all the display elements will be the same color and the combined tip position display 136 and orientation display 138 illustrated in FIG. 8 will indicate that the 25 drill bit 110 is in the proper orientation and that the tool tip 112 is in the correct position so that the procedure may begin. In addition, it is possible that the display could also flash individual display elements or groups of display elements or change the color intensity or brightness of these display elements to show the proper position and/or orientation has been achieved. As noted previously, the display elements that comprise the display screen 122 can be LED's or an LCD screen or similar display device comprising a series of pixels.

As an alternate embodiment, it may be desired to be able to show the depth and progress of the procedure also on the display 122. This can be done either by the use of various colors to identify various stages of both orientation position and depth or alternatively by the use of a third display element, a depth display 200, as shown in FIG. 9. For a multicolor display, the unaligned tip position display 136 and the unaligned orientation display 138 as shown in FIGS. 1, 2, 3, 5, 6 and 7 can be red. The properly positioned tip position display 136 and the properly positioned orientation display 138 can be green. As the tool is operated and the drill 100 proceeds ahead on the properly positioned orientation display 138 will remain green but the properly positioned tip position display 136 will change color to an alternative color such as yellow indicating that the end position or depth has not yet been reached. When the proper depth as indicated by presurgical planning is reached the color of the properly positioned tip position display 136 will change color to green indicating the final depth has been reached.

As shown in FIG. 9, the display 122 can also include the depth display 200. The depth display can be a single LED or pixel that will move to the center of the right edge of the display 122 as the drill 100 reaches the proper depth. The exact position of the end point is not important so long as the surgeon knows the location of the proper depth end point. For instance, it may be desired to provide more sensitivity for the depth measurement and the location of the end point will be the bottom right most LED or pixel.

As indicated above, the tracking and display unit 120 may also include the first function button 130 and the second function button 132. These function buttons can serve a number of functions pending upon the status of the tracking and display unit 120. For instance, it will be necessary to calibrate the position and orientation of the tool tip 112 and the drill bit 110 relative to the tracking and display unit 120 and relative to the surgical navigation system. In this regard the first function button 130 can be utilized to signal the system to begin a calibration procedure so that the surgical navigation can properly calibrate the location and orientation of both the surgical drill 100 and the tool tip 112 to the location and orientation of the attached tracking and display unit 120. An alternative function for the first function button 130 after calibration has been completed could be to simplify the display shown on the display screen 122. For instance, instead of showing the tip position display 136, the orientation display 138, and the depth display 200 on the screen at the same time, the display 122 could be programmed such that only the tip position 136 is initially shown in display 122 so that the display will be simplified for the surgeon operating the surgical drill 100. After the tip position display 136 is properly aligned, the surgeon can press the first function button 130 and the orientation display 138 then will appear on the display 122. The second function button 132 can also have similar functionality as well as serving as an off/on switch for the tracking and display unit 120. Both the first function button and the second function button can cooperate with the surgical navigation system and act as mouse buttons so the surgeon does not have to manipulate a separate computer mouse or instruct someone else to manipulate the mouse.

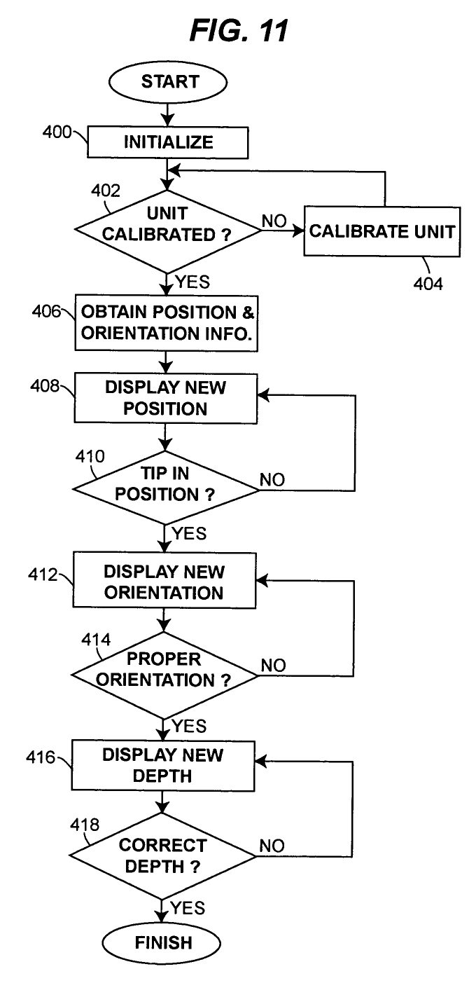

Turning now to FIG. 11, which is a flow diagram of the method of the present invention. The surgical navigation system 500 will start performing the method of the present invention by passing control to a block 400 that initializes the tracking and display unit 120 relative to the surgical navigation system 500. As part of the initialization process, the tracking and display unit 120, in response to instructions from the surgical navigation system 500, will indicate to the user possibly either by a visual display on the display unit 122 or by repeated flashing of the status light 134 that the tracking and display unit 120 and drill 100 combination must be calibrated prior to use. A block 402 determines whether or not the tracking and display unit 120 has been properly calibrated relative to the surgical navigation system 500 and relative to the particular drill bit 110 that has been inserted into the drill chuck 108. If the device has not been calibrated, control passes to a block 404 that performs the calibration of the tool in a known manner and returns control back to the block 402 that determines whether the calibration has been successful. If calibration is successful, control passes to a block 406 that identifies the predetermined position of the tool tip 112 and predetermined orientation of the drill bit 110 either from the pre-surgical planning that has been done within the surgical navigation system 500 or from other imageless position determination methods known to those in the art. This position and orientation information is then passed from the surgical navigation system 500 to memory unit (not shown) within the computer 504. Proper calibration of the tool tip 112 enables the surgical navigation system 500 to instruct the tracking and display unit 120 to properly display the proper information for position for the tool tip 112 and the orientation of the drill bit 110. Upon receiving the predetermined position and orientation information for the tool tip 112 and the drill bit 110 control passes to a block 408 that instructs the display of the position of the tool tip 112 on display 122 relative to the predetermined x, y, z position for the tool tip 112. The display block 408 will continuously display the current position of the tool tip 112 and a block 410 will determine whether or not the tool tip 112 is in the proper predetermined position. If the tool tip is not in the proper predetermined position control will branch back to display block 408 which again will repeat the cycle until block 410 determines that the tool tip 112 is in a proper x, y, z location. At this time, control then passes to a block 412, which displays the orientation of the drill bit 110 and then passes control to a block 414, which determines whether the orientation of the drill bit 110 is in the proper orientation. If the orientation does not match the predetermined orientation, then control branches back to the display block 412 that continuously displays the position of the orientation of the drill bit 110 on the display 122. When the block 414 determines that the drill bit 110 is in the proper orientation control then passes to a block 416 that displays the depth information on the display 122. Control then passes to a block 418 that determines whether or not the x, y, z position of the tool tip 112 is at the proper predetermined depth for the termination of the surgical procedure. If the tool tip 112 is not at the proper depth, control passes back to a display box 416 that shows the depth display on the display 122 either by color change or by a separate depth display 200 that the depth has not been yet reached. When a proper depth is reached, the block 418 will indicate the information to the surgeon at which time the procedure is completed and the process ends.

It will be appreciated that the process step of the block 406 can be conducted prior to the step of determining if the tool has been calibrated. Also, the process steps of the blocks 408 and 410, the blocks 412 and 414, and the blocks 416 and 418 can be performed in any order or essentially simultaneously so that the user perceives the information displayed on the display 122 is real time information that changes as the tool tip 112 is moved by the user.

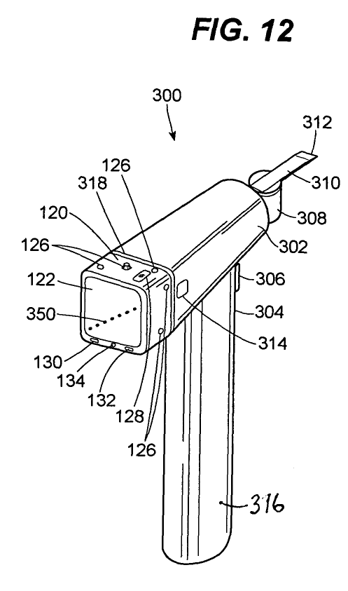

FIG. 12 shows an additional surgical power tool, a surgical saw 300 having a tool body 302, a tool handle 304, a control switch 306, a blade mounting assembly 308, and a saw blade 310. The saw blade 310 has saw teeth 312. The tool body 302 has a release button 314 that operates in a manner similar to release button 114. The tool handle 304 is designed to accommodate a tool battery pack 316. The tool body 302 has a distal end 318. The tracking and display unit 120 is attached to the distal end 318 in the same manner as to the distal end 118 of the surgical drill 100. The tracking and display unit 120 is in all respects similar to the tracking and display unit 120 used with the surgical drill 100. As shown in FIG. 12, the display 122 for use with a surgical saw includes a saw position display 350. The saw position display could be a single point in a manner similar to the tip position display 136. As shown, the saw position display 350 is a line of LED's or pixels that indicates the relative position of the saw teeth 312 to the pre-surgical planning cut location.

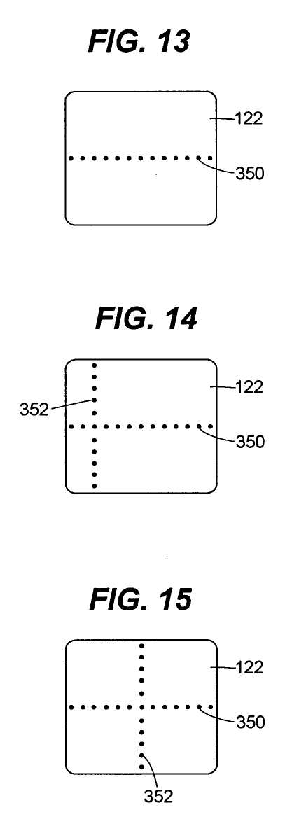

FIGS. 12 to 15 diagrammatically shows the possible operation of the display 122 when configured for use with a surgical saw 300. In FIG. 12 the display indicates that the saw teeth 312 are below the location of the site of the planned cut. Since the saw blade 310 is a plane, the display 122 shows the relative location of the plane of the saw blade 310 to the plane of the planned cut location. As shown in FIG. 12, the saw 300 must be rotated in a counter clockwise fashion. FIG. 13 shows the display 122 when the saw 300, the saw blade 310 and the saw teeth are placed in the proper position and rotational orientation. FIG. 14 shows an added display 352 as a vertical line of LED's or pixels that assist the surgeon in positioning the saw blade 310 so that the x, y, z position is correct, the rotational orientation is correct and the angle of attack is correct. As shown in FIG. 14, the saw 300 must be tilted upwards so the angle of attack of the saw blade 310 is on the proper plane. As the saw 300 is tilted upwards, and the position and rotational component are held constant, the display 122 will follow the upwards tilt with line of LED's 352 moving from the left side of the display 122 to the center of the display 122 as shown in FIG. 15.

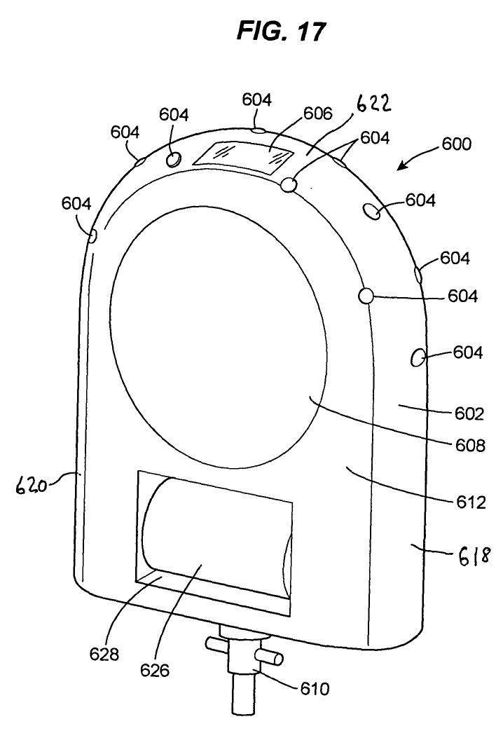

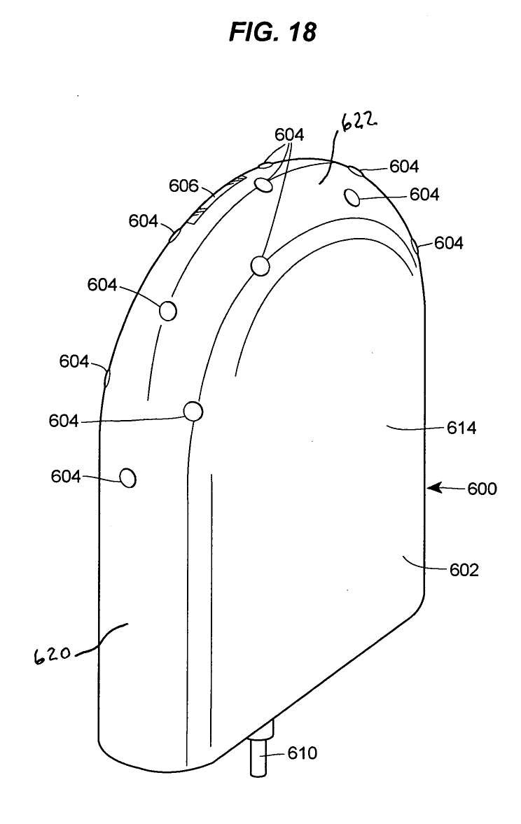

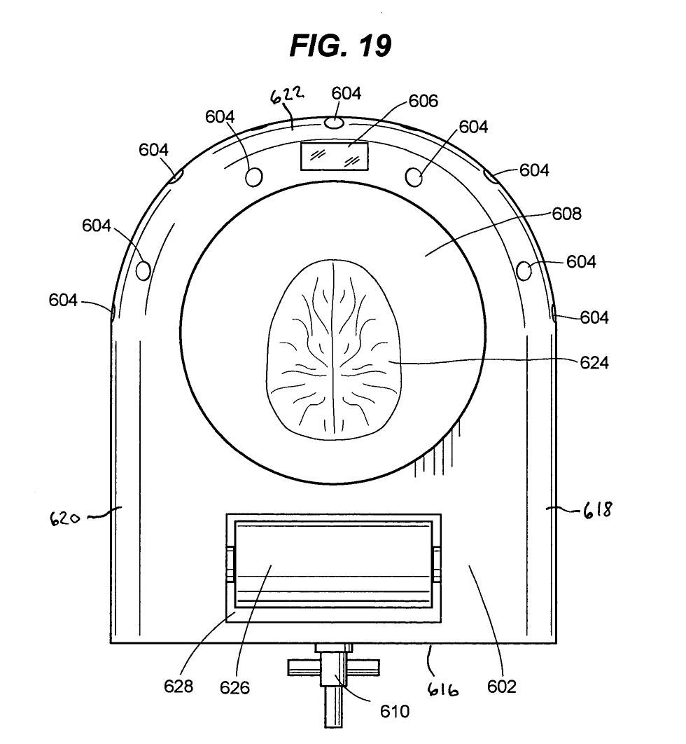

FIGS. 17, 18, and 19 show an alternative display unit 600 of the present invention. The display unit 600 has a unit body 602 having a front 612, a rear 614, a base 616, sides 618 and 620 and a top 622. Mounted on the sides 618 and 620 and the top 622 of the unit body 602 are a series of LED's 604, and a transceiver 606. A display screen 608 is mounted on the front 612. The display screen 608 is transparent and covers LED array (not shown). The display screen 608 operates in a manner similar to display screen 122 described above and may include an image 624. A docking pin 610 is attached to the base 616 of the unit body 602. The docking pin 610 is similar to the docking pin 152 described above. A battery 626 powers the display unit 600. The battery 626 is inserted into a battery holder 628.

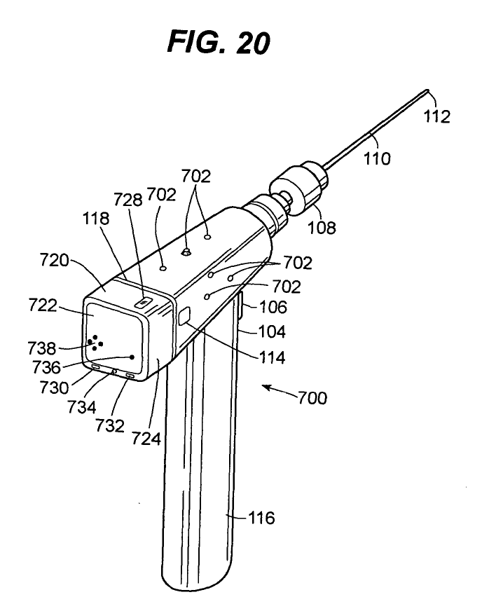

FIG. 20 shows a surgical drill 700 that has a series of built in LED's 702. It is also within the scope of the present invention for the surgical drill 700 to have a tracking unit attached to the exterior of the surgical drill 700 in some fashion, such as by an attachment adapter or by an adapter that is designed into the surface of the surgical drill 700. A display unit 720 is attached to the distal end 118 of the surgical drill 700 in a manner similar to the attachment of the display unit 120 to the drill 100 described above. The display unit 720 includes a display screen 722 similar to the display screen 122, a display unit body 724 similar to the display unit body 124, and a transceiver window 728 through which a transceiver contained within the display unit 720 can communicate with the surgical navigation system 500. Also shown in FIG. 20 are optional function buttons 730 and 732 and optional status light 734. In certain environments and systems, it will not be necessary for the display unit 720 to actively interact with the surgical navigation system 500, but merely be a passive display element similar to a computer monitor. Depending on the nature of the display screen 722, the display screen 722 can also display status messages and other information that the surgical navigation system 500 sends to the display unit 720 to provide information to the user. The display screen 722 also shows a position display 736 and an orientation display 738.

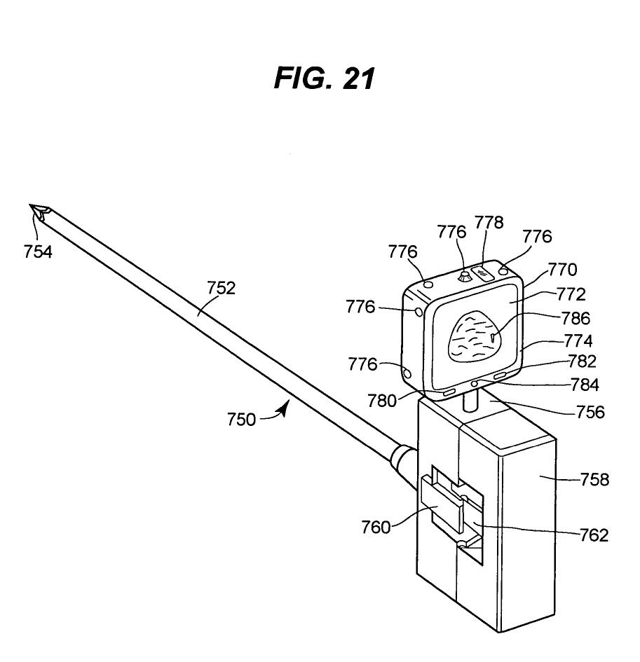

FIG. 21 shows a non-powered hand held surgical tool, a biopsy device 750, that has a sleeve 752 surrounding a biopsy needle (not visible) having a tip 754. The biopsy device 750 has a first handle 756 and a second handle 758. The first handle 756 is directly attached to the sleeve 752 and the second handle 758 is directly attached to the biopsy needle and the tip 754. The first handle 756 has a pair of first tabs 760 that interlock with a pair of second tabs 762 on the second handle 758 to hold the biopsy needle and tip 754 in place within the shaft 752. Attached to the first handle 756 is a display unit 770 that is similar to the display unit 120. The display unit 770 includes a display screen 772 capable of displaying a display image 786. The display unit 770 also includes a display unit body 774, a series of LED's 776 and a transceiver 778 to communicate with the surgical navigation system 500. Below the display screen 774 are first and second function buttons 780 and 782. A status light 784 is located between the first end second function buttons 780 and 782. Devices similar to the biopsy device 750 can also be used along with the display unit of the present invention and in accordance with the present invention. These similar devices include trocars, drill guides, laparoscopic tools, and the like.

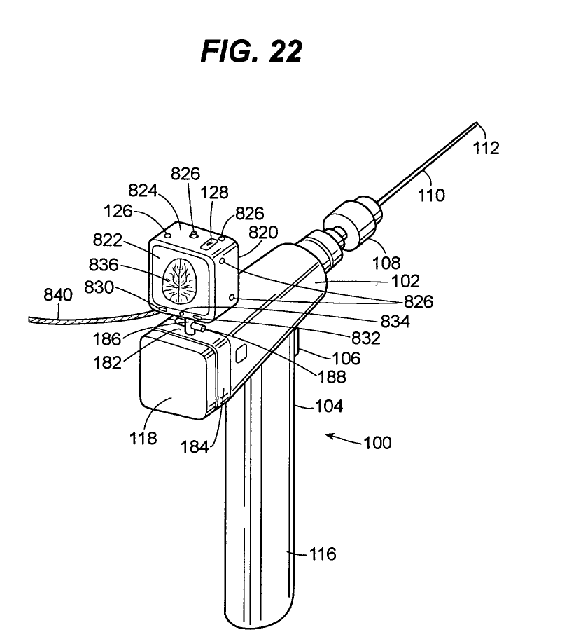

FIG. 22 shows a still further embodiment of the present invention mounted to a surgical drill 100 in a manner similar to that described with reference to FIG. 10. The display unit 820 has a display unit body 824 and a display screen 822. The display screen 822 is capable of displaying images such as a display image 836, which is a portion of the image visible on the monitor 506. The display unit 820 also has a series of LED's 826 that function in a manner similar to that described with reference to LED's 126. The display unit 820 includes first and second function buttons 830 and 832 and a status light 834.

The display unit 820 is connected to the surgical navigation system 500 by a hard wired communications link 840 can be used in place of the wireless communications link utilized by the display unit 120 and the transceiver 512 in the surgical navigation system 500. Depending on the nature and volume of data to be communicated between the surgical navigation system 500 and the various display units such as the display unit 120 or 820, either or both a wireless communication link and a hard wired communication link may be used.

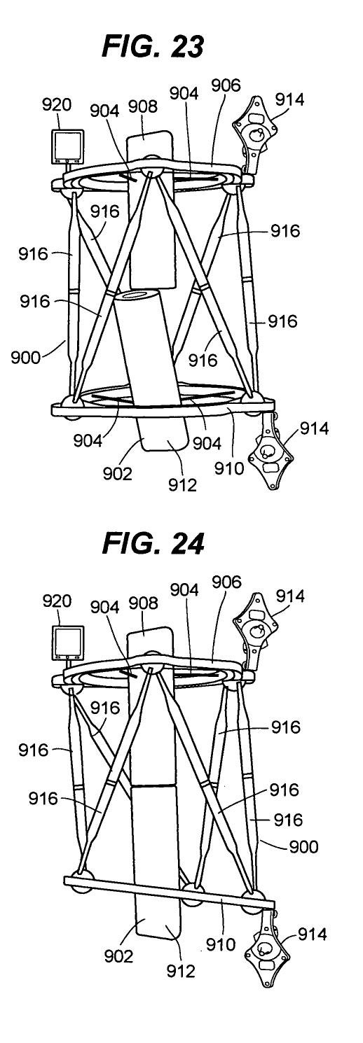

It is appreciated that in addition to using the method of the present invention to guide a tool or instrument to a specific location, the method of the present invention can also used with tools that require manipulation to position a tool, jig, guide, or instrument relative to a patient's anatomy. For instance, as shown in FIGS. 23 and 24 a display unit 920 similar in functionality to the previously described display units, particularly display unit 720, can be attached to an external fixator unit 900 that has been previously attached to a broken bone 902 within a patient's leg (not shown for clarity). The fixator 900 is attached to the bone 902 using a series of rods or pins 904 (not all are shown) in accordance with well-known techniques. An upper fixator ring 906 is firmly attached to an upper bone piece 908 and a lower fixator ring 910 is attached to a lower bone piece 912. A tracking device 914 that can be seen and tracked by the surgical navigation system 500 is attached to both the upper fixator ring 906 and the lower fixator ring 912. A series of adjustable rods 916 connect the upper fixator ring 906 to the lower fixator ring 910. The adjustable rods 916 can be adjusted to change the relative length of each of the adjustable rods 916 to change to position and orientation of the upper fixator ring 906 relative to the lower fixator ring 910. Because the fixator rings 906 and 910 are firmly attached to the bone pieces 908 and 912, any movement of the fixator ring also moves the bone attached to that fixator ring. By adjusting some or all of the adjustable rods 916, the bone pieces 908 and 912 can be brought into proper alignment as shown in FIG. 24. The user can view the related anatomical information on the display unit 920 as the fixator 900 is manipulated by adjusting the adjustable rods 916 to bring the bone pieces 908 and 912 into proper position and alignment. The related anatomical information that is displayed on the display unit 920 can include position information, orientation information, kinematics information relative to the target bone or joint and any similar information.

While the tracking unit and display 120 is particularly useful for power surgical tools such as drills, saws, and the like, and non-power hand tools or instruments such as biopsy needles, as disclosed herein, it can be used with a wide variety of other surgical tools and instruments, both powered and non-powered, such as, screw drivers, reamers, pointers, aspirators and the like where the surgeon will benefit from a display placed directly on the tool so that the surgeon can maximize the benefits of the surgical navigation system 500 while using the tool without having to look away from the surgical site.

### Industrial Applicability

Numerous modifications to the present invention will be apparent to those skilled in the art in view of the foregoing description. Accordingly, this description is to be construed as illustrative only and is presented for the purpose of enabling those skilled in the art to make and use the invention and to teach the best mode of carrying out same. The exclusive rights to all modifications, which come within the scope of the appended claims, are reserved.

1. A display unit for use with a surgical instrument to be manipulated relative to a patient's anatomy having a surgical instrument body and a tracking unit associated with the surgical instrument capable of communicating with a surgical navigation system comprising:
a communication link to communicate with the surgical navigation system;
a screen to display information received from the surgical navigation system; and
an attachment device to attach the display unit to the surgical instrument body.

2. The display unit of claim 1 wherein the surgical instrument has a tool tip and the screen displays the position of the tool tip relative to a predetermined position.

3. The display unit of claim 2 wherein the screen also displays the orientation of the surgical instrument relative to a predetermined orientation.

4. The display unit of claim 2 wherein the screen also displays the depth of the tool tip within a surgical site relative to a predetermined depth.

5. The display unit of claim 1 wherein the display is an array of light emitting diodes.

6. The display unit of claim 1 wherein the display unit can be attached to the surgical instrument by a quick release coupling.

7. The display unit of claim 1 wherein the display unit includes a function button.

8. The display unit of claim 1 wherein the display unit is attached to the surgical instrument so that the predetermined position and the screen can be viewed at the same time.

9. The display unit of claim 1 wherein the display unit displays a diagnostic image.

10. The display unit of claim 9 wherein the display unit also displays an image of the instrument relative to the diagnostic scan image.

11. The display unit of claim 1 wherein the display unit also displays an image of the instrument relative to an image of an implant.

12. The display unit of claim 1 wherein the display unit displays a subset of the information displayed on a monitor associated with the surgical navigation system.

13. The display unit of claim 1 wherein the display unit displays information provided by the surgical instrument.

14. The display unit of claim 1 wherein the communication link is wireless.

15. A display unit for use with a surgical navigation system comprising:
a body;
a screen capable of displaying data;
a tracking unit associated with the body that can be tracked by the surgical navigation system;
communications link capable of sending and receiving data to and from the surgical navigation system; and
an attachment device to attach the display unit to a body of a surgical instrument to be manipulated relative to a patient's anatomy,
wherein the screen can display information relating to the position of the surgical instrument relative to the patient's anatomy.

16. The display unit of claim 15 wherein the screen also displays the orientation of the surgical instrument relative to a predetermined orientation.

17. The display unit of claim 15 wherein the surgical instrument has a tool tip and the screen also displays the depth of the tool tip within a surgical site relative to a predetermined depth.

18. The display unit of claim 15 wherein the display is an array of light emitting diodes.

19. The display unit of claim 15 wherein the display unit is integral with the surgical instrument body.

20. The display unit of claim 15 wherein the display unit can be attached to the surgical instrument by a quick release coupling.

21. The display unit of claim 15 wherein the display unit includes a function button.

22. The display unit of claim 15 wherein the display unit is attached to the surgical tool so that the predetermined position and the screen can be viewed at the same time

23. The display unit of claim 15 wherein the communication link is wireless.

24. The display unit of claim 15 wherein the display unit displays a diagnostic image.

25. The display unit of claim 24 wherein the display unit also displays an image of the instrument relative to the diagnostic scan image.

26. The display unit of claim 15 wherein the display unit displays information provided by the surgical instrument.

27. The display unit of claim 15 wherein the display unit also displays an image of the instrument relative to an image of an implant.

28. A surgical tool comprising;
a tool body;
a tool tip;
a communications link associated with the tool body capable of sending and receiving data to and from a surgical navigation system;
a display unit associated with the tool body wherein the display unit is in communication with the surgical navigation system; and
a tracking unit associated with the tool body such that the surgical tool can be tracked by the surgical navigation system,
wherein the display unit has a screen capable of displaying information relating to the position of the tool tip relative to a predetermined position.

29. The surgical tool of claim 28 wherein the screen also displays the orientation of the surgical tool relative to a predetermined orientation.

30. The surgical tool of claim 28 wherein the screen also displays the depth of the tool tip within a surgical site relative to a predetermined depth.

31. The surgical tool of claim 28 wherein the display is an array of light emitting diodes.

32. The surgical tool of claim 28 wherein the display unit is integral with the tool body.

33. The surgical tool of claim 28 wherein the display unit includes a function button.

34. The surgical tool of claim 28 wherein the display unit is associated with the surgical tool so that the predetermined position and the screen can be viewed at the same time.

35. The surgical tool of claim 28 wherein the surgical tool is a power tool.

36. The surgical tool of claim 35 wherein the power tool is a surgical drill.

37. The surgical tool of claim 35 wherein the power tool is a surgical saw.

38. The surgical tool of claim 28 wherein the surgical tool is a non-powered hand instrument.

39. The surgical tool of claim 38 wherein the non-powered hand instrument is a biopsy needle.

40. The surgical tool of claim 28 wherein the communications link is wireless

41. A method of positioning a tool tip of a surgical tool relative to a predetermined position, the method comprising the steps of;
viewing the position of the tool tip relative to the predetermined position on a display unit attached to the surgical tool, the display unit receiving data on the location of the tool tip from a surgical navigation system; and
guiding the tool tip to the predetermined position while viewing the both the predetermined position and the display unit.

42. The method of claim 41 wherein the display unit also displays the orientation of the surgical tool relative to a predetermined orientation.

43. The method of claim 41 wherein the display unit also displays the depth of the tool tip within a surgical site relative to a predetermined depth.

44. The method of claim 41 wherein the display unit is an array of light emitting diodes.

45. The method of claim 41 wherein the display unit is integral with the surgical tool body.

46. The method of claim 41 wherein the surgical tool is a power tool.

47. The method of claim 46 wherein the power tool is a surgical drill.

48. The method of claim 46 wherein the power tool is a surgical saw.

49. The method of claim 41 wherein the surgical tool is a non-powered hand instrument.

50. The method of claim 49 wherein the non-powered hand instrument is a biopsy needle.

51. A method of positioning a surgical device relative to a patient's anatomy, the method comprising the steps of;
viewing information representing the position of the surgical device relative to the patient's anatomy on a display unit attached to the surgical device, the display unit receiving data about the location of the surgical device and data about the location of the patient's anatomy from a surgical navigation system; and
manipulating the surgical device relative to the patient's anatomy using data displayed on the display unit while viewing the both the patient's anatomy and the display unit.

52. The method of claim 51 wherein the information on the display unit includes kinematics information.

53. The method of claim 51 wherein the surgical device is a power tool.

54. The method of claim 53 wherein the power tool is a surgical drill.

55. The method of claim 53 wherein the power tool is a surgical saw.

56. The method of claim 51 wherein the surgical device is a non-powered hand instrument.

57. The method of claim 56 wherein the non-powered hand instrument is an external fixator device.

58. The method of claim 56 wherein the non-powered hand instrument is a biopsy needle.
